(19) United States
(12) Patent Application Publication
(10) Pub. No.: US 2026/0207448 A1
(43) Pub. Date: Jul. 23, 2026

(54) PERSONAL CARE COMPOSITION COMPRISING A VOLATILE ALKANE MIXTURE AND NON-VOLATILE OILS

(71) Applicant: MOMENTIVE PERFORMANCE MATERIALS INC., Niskayuna, NY (US)

(72) Inventors: Linda WEI, Guangdong (CN); Amar PAWAR, Tarrytown, NY (US); Benjamin FALK, Tarrytown, NY (US); Roxanne SMITH, Oxfordshire (GB); Mana TAMAMI, Tarrytown, NY (US)

(73) Assignee: MOMENTIVE PERFORMANCE MATERIALS INC., Niskayuna, NY (US)

(21) Appl. No.: 19/135,766

(22) PCT Filed: Dec. 5, 2022
(86) PCT No.: PCT/CN2022/136539

§ 371 Date: Jun. 4, 2025

(51) Int. Cl. A61K 8/31; A61K 8/29; A61K 8/37; A61K 8/42; A61K 8/891; A61K 8/892; A61K 8/92; A61Q 1/04; A61Q 5/12; A61Q 17/04; A61Q 19/00

(52) U.S. Cl. CPC A61K 8/31; A61K 8/29; A61K 8/37; A61K 8/42; A61K 8/891; A61K 8/892; A61K 8/922; A61Q 1/04; A61Q 5/12; A61Q 17/04; A61Q 19/004

(57) ABSTRACT

Personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile oils selected from the group consisting of a silicone oil, a hydrocarbon oil, an organic oil, a fluorinated oil, and combinations thereof.

### FIELD OF THE DISCLOSURE

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile oils selected from the group consisting of a silicone oil, a hydrocarbon oil, a fluorinated oil, an organic oil, and combinations thereof. The present disclosure also relates to linear alkanes comprising about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture. The present disclosure further relates to a non-volatile oil selected from the group consisting of a conditioning agent, an emollient, a sunscreen oil, a natural oil, and combinations thereof.

### BACKGROUND

Conditioning agents, emollients, sunscreen oils, and natural oils and esters are commonly used in personal care applications as functioning oils to provide moisturized soft feel for hair, soft moisturization to skin, protection from UV rays, and nourishment to skin respectively. However, the feel of such non-volatile oils can be heavy, greasy and tacky, and thus is undesired. To achieve light and dry perception, these non-volatile oils need to be delivered through volatile carrier solvents to skin. Currently used volatile solvents include silicone-based solvents such as cyclopentasiloxane and methyl trimethicone, or hydrocarbon solvents such as isododecane and isohexadecane. The silicone-based solvents present challenges due to their non-biodegradibility. The hydrocarbon solvents such as isododecane (IDD) can flash off the skin too quickly leaving the skin dry and uncomfortable. The ester-based organic solvents or other hydrocarbon solvents, such as isohexadecane, are non-volatile and do not satisfy the need of a non-greasy and non-tacky feel. Thus, there is a need to develop personal care formulations comprising a solvent which can maintain functions and benefits of non-volatile oils, and also provide dry, non-greasy, non-tacky, and soft sensory feel. The personal care compositions of the present disclosure provides these sensory benefits while maintaining the functional benefits of the non-volatile oils.

### BRIEF SUMMARY OF THE DISCLOSURE

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile oils selected from the group consisting of a silicone oil, a hydrocarbon oil, a fluorinated oil, an organic oil, and combinations thereof.

In some aspects, the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

In some aspects, the non-volatile oil is silicone with viscosity from about 10 cP to about 200,000,000 cP at 25° C.

In some aspects, the non-volatile oil is a silicone gum selected from the group consisting of polyalkylsiloxane, polyarylsiloxane, polyalkylsilanol, and combinations thereof.

In some aspects, the non-volatile oil is an organic oil selected from the group consisting of a C₈-C₄₀ fatty alcohol, an ester of a C₇-C₃₉ fatty acid, an ester of a C₈-C₄₀ fatty alcohol, polyisobutene, a wax, a plant oil including jojoba oil, argen oil, coconut oil, almond oil, castor oil, and avocado oil, a mineral oil, a synthetic oil, and combinations thereof.

In some aspects, the non-volatile oil is an organic sunscreen ingredient selected from the group consisting of avobenzone, octisalate, oxybenzone, homosalate, octocrylene, octyl methoxycinnamate, 2-ethoxyethyl p-methoxycinnamate, ethylhexyl salicylate, ethylhexyl methoxycinnamate, and combinations thereof.

In some aspects, the non-volatile oil is selected from the group consisting of a conditioning agent, an emollient, a sunscreen oil, a natural oil, and combinations thereof.

### BRIEF DESCRIPTION OF THE FIGURE

The accompanying FIGURE, which is incorporated herein, forms part of the specification and illustrates embodiments of the present disclosure. Together with the description, the FIGURE further serves to explain the principles of and to enable a person skilled in the relevant art(s) to make and use the disclosed embodiments. The FIGURE is intended to be illustrative, not limiting.

FIG. 1 describes a sensory panel study for Formulation Examples 2a and 2b.

### DETAILED DESCRIPTION

As used above, and throughout the description, the following terms, unless otherwise indicated, shall be understood to have the following meanings.

Unless stated otherwise, the terms “a” and “an” and “the” and similar references used in the context of describing a particular aspect of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” encompasses the range of experimental error that occurs in any measurement.

The term, “hydrocarbon” as used herein refers to any chemical structure containing hydrogen atoms and carbon atoms.

The term “alkyl” means any monovalent, saturated straight chain or branched chain hydrocarbon group; and the term “alkane” means organic compounds consisting entirely of single-bonded carbon and hydrogen atoms.

Representative examples of alkyls include, but are not limited to, methyl, ethyl, propyl and isobutyl. Examples of alkanes include, but are not limited to, methane, ethane, propane and isobutane.

The term “aryl” includes any aromatic hydrocarbon from which one hydrogen atom has been removed. Specific, non-limiting examples of aryl groups include phenyl and naphthalenyl.

The term “linear C₉-C₁₈ alkanes” means linear alkanes having 9 to 18 alkanes. Examples of linear C₉-C₁₈ alkanes include, but are not limited to, n-decane, n-dodecane, and n-tetradecane.

The term “branched C₁₀-C₁₄ alkane” means branched alkanes having 10 to 14 carbon atoms. Examples of branched C₁₀-C₁₄ alkane include, but are not limited to, isodecane and isododecane.

Other than in the working examples or where otherwise indicated, all numbers expressing amounts of materials, reaction conditions, time durations, quantified properties of materials, and so forth, stated in the specification and claims are to be understood as being modified in all instances by the term “about”.

It will be understood that any numerical range recited herein includes all sub-ranges with that range and any combination of the various endpoints of such ranges or sub-ranges.

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

### I. Volatile Alkane Mixtures

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile oils selected from the group consisting of a silicone oil, a hydrocarbon oil, a fluorinated oil, an organic oil, and combinations thereof.

In some aspects, the volatile alkane mixture comprises about 10% to about 90% by weight of linear C₉-C₁₈ alkanes, about 15% to about 90% by weight of linear C₉-C₁₈ alkanes, about 20% to about 90% by weight of linear C₉-C₁₈ alkanes, about 25% to about 85% by weight of linear C₉-C₁₈ alkanes, about 30% to about 85% by weight of linear C₉-C₁₈ alkanes, about 35% to about 80% by weight of linear C₉-C₁₈ alkanes, about 40% to about 80% by weight of linear C₉-C₁₈ alkanes, about 45% to about 75% by weight of linear C₉-C₁₈ alkanes, about 50% to about 75% by weight of linear C₉-C₁₈ alkanes, about 55% to about 70% by weight of linear C₉-C₁₈ alkanes, or about 60% to about 70% by weight of linear C₉-C₁₈ alkanes relative to the total weight of the alkane mixture.

In some aspects, the volatile alkane mixture comprises about 30% to about 85% by weight of linear C₉-C₁₈ alkanes relative to the total weight of the alkane mixture.

In some aspects, the volatile alkane mixture comprises about 50% to about 75% by weight of linear C₉-C₁₈ alkanes relative to the total weight of the alkane mixture.

In some aspects, the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

In some aspects, the linear alkanes comprise about 0% to about 5% by weight of n-decane, about 1% to about 4% by weight of n-decane, or about 2% to about 3% by weight of n-decane relative to the total weight of the alkane mixture. In some aspects, the n-decane is about 0% by weight, about 1% by weight, about 2% by weight, about 3% by weight, about 4% by weight, or about 5% by weight relative to the total weight of the alkane mixture. In some aspects, the n-decane is about 1% by weight relative to the total weight of the alkane mixture. In some aspects, the n-decane is about 5% by weight relative to the total weight of the alkane mixture.

In some aspects, the linear alkanes comprise about 50% to about 80% by weight of n-dodecane, about 55% to about 75% by weight of n-dodecane, about 60% to about 70% by weight of n-dodecane, or about 65% to about 70% by weight of n-dodecane relative to the total weight of the alkane mixture. In some aspects, the n-dodecane is about 50% by weight, about 55% by weight, about 60% by weight, about 65% by weight, about 70% by weight, about 75% by weight, or about 80% by weight relative to the total weight of the alkane mixture. In some aspects, the n-dodecane is about 60% by weight relative to the total weight of the alkane mixture. In some aspects, the n-dodecane is about 65% by weight relative to the total weight of the alkane mixture. In some aspects, the n-dodecane is about 70% by weight relative to the total weight of the alkane mixture.

In some aspects, the linear alkanes comprise about 1% to about 5% by weight of n-tetradecane, about 2% to about 4% by weight of n-tetradecane, or about 3% to about 4% by weight of n-tetradecane relative to the total weight of the alkane mixture. In some aspects, the n-tetradecane is about 1% by weight, about 2% by weight, about 3% by weight, about 4% by weight, or about 5% by weight relative to the total weight of the alkane mixture. In some aspects, the n-tetradecane is about 1% by weight relative to the total weight of the alkane mixture. In some aspects, the n-tetradecane is about 5% by weight relative to the total weight of the alkane mixture.

In some aspects, the linear alkanes comprise about 0% to about 5% by weight of n-hexadecane, about 1% to about 4% by weight of n-hexadecane, or about 2% to about 3% by weight of n-hexadecane relative to the total weight of the alkane mixture. In some aspects, the n-hexadecane is about 0% by weight, about 1% by weight, about 2% by weight, about 3% by weight, about 4% by weight, or about 5% by weight relative to the total weight of the alkane mixture. In some aspects, the n-hexadecane is about 0% by weight relative to the total weight of the alkane mixture. In some aspects, the n-hexadecane is about 1% by weight relative to the total weight of the alkane mixture. In some aspects, the n-hexadecane is about 5% by weight relative to the total weight of the alkane mixture.

In some aspects, the linear alkanes comprise about 0% to about 5% by weight of n-octadecane, about 1% to about 4% by weight of n-octadecane, or about 2% to about 3% by weight of n-octadecane relative to the total weight of the alkane mixture. In some aspects, the n-octadecane is about 0% by weight, about 1% by weight, about 2% by weight, about 3% by weight, about 4% by weight, or about 5% by weight relative to the total weight of the alkane mixture. In some aspects, the n-octadecane is about 0% by weight relative to the total weight of the alkane mixture. In some aspects, the n-octadecane is about 1% by weight relative to the total weight of the alkane mixture. In some aspects, the n-octadecane is about 5% by weight relative to the total weight of the alkane mixture.

In some aspects, the linear alkanes are of plant origin. In some aspects, the linear alkane of plant origin is n-decane. In some aspects, the linear alkane of plant origin is n-dodecane. In some aspects, the linear alkane of plant origin is n-tetradecane. In some aspects, the linear alkane of plant origin is n-hexadecane. In some aspects, the linear alkane of plant origin is n-octadecane.

In some aspects, the linear alkanes are free of alkanes with odd number carbon atoms. In some aspects, the linear alkane is n-decane. In some aspects, the linear alkane is n-dodecane. In some aspects, the linear alkane is n-tetradecane. In some aspects, the linear alkane is n-hexadecane. In some aspects, the linear alkane is n-octadecane.

In some aspects, the volatile alkane mixture comprises about 10% to about 90% by weight of branched C₁₀-C₁₄ alkanes, about 10% to about 85% by weight of branched C₁₀-C₁₄ alkanes, about 10% to about 80% by weight of branched C₁₀-C₁₄ alkanes, about 15% to about 75% by weight of branched C₁₀-C₁₄ alkanes, about 15% to about 70% by weight of branched C₁₀-C₁₄ alkanes, about 20% to about 65% by weight of branched C₁₀-C₁₄ alkanes, about 20% to about 60% by weight of branched C₁₀-C₁₄ alkanes, about 25% to about 55% by weight of branched C₁₀-C₁₄ alkanes, about 25% to about 50% by weight of branched C₁₀-C₁₄ alkanes, about 30% to about 45% by weight of branched C₁₀-C₁₄ alkanes, or about 30% to about 40% by weight of branched C₁₀-C₁₄ alkanes relative to the total weight of the alkane mixture. In some aspects, the volatile alkane mixture comprises about 15% to about 70% by weight of branched C₁₀-C₁₄ alkanes relative to the total weight of the alkane mixture. In some aspects, the volatile alkane mixture comprises about 25% to about 50% by weight of branched C₁₀-C₁₄ alkanes relative to the total weight of the alkane mixture.

### II. Non-Volatile Oils

In some aspects, the non-volatile oils described above are selected from the group consisting of a silicone oil, a hydrocarbon oil, a fluorinated oil, an organic oil and combinations thereof. In some aspects, the non-volatile oil is a silicone oil. In some aspects, the non-volatile oil is a hydrocarbon oil. In some aspects, the non-volatile oil is a fluorinated oil. In some aspects, the non-volatile oil is an organic oil.

In some aspects, the non-volatile oil is silicone with viscosity at 25° C. from about 10 cP to about 200,000,000 cP, about 10,000 cP to about 150,000,000 cP, about 100,000 cP to about 100,000,000 cP, about 1,000,000 cP to about 50,000,000 cP.

In some aspects, the non-volatile oil is a silicone gum selected from the group consisting of polyalkylsiloxane, polyarylsiloxane, polyalkylsilanol, and combinations thereof. In some aspects, the silicone gum is polyalkylsiloxane. In some aspects, the silicone gum is polyarylsiloxane. In some aspects, the silicone gum is polyalkylsilanol. Examples of such gums include but are not limited to siloxanes with INCI names (International Nomenclature Cosmetic Ingredient) dimethicone, dimethiconol and are commercially available under Silsoft 1215, CF1251, SE30 gum, SF1236, SF1276 from Momentive Performance Materials.

In some aspects, the non-volatile oil is selected from the group consisting of an amino modified silicone, a polyether modified silicone, a phenyl modified silicone, and combinations thereof. In some aspects, the non-volatile oil is an amino modified silicone. In some aspects, the non-volatile oil is a polyether modified silicone. In some aspects, the non-volatile oil is a phenyl modified silicone.

In some aspects, the non-volatile oil is a phenyl modified silicone selected from the group consisting of phenyltrimethicone, triphenyl dimethylvinyl disiloxane, phenylpropyldimethylsiloxysilicate, and combinations thereof. In some aspects, the phenyl modified silicone is phenyltrimethicone. In some aspects, the phenyl modified silicone is triphenyl dimethylvinyl disiloxane. In some aspects, the phenyl modified silicone is phenylpropyldimethylsiloxysilicate.

In some aspects, the non-volatile oil is an organic oil selected from the group consisting of a C₈-C₄₀ fatty alcohol, an ester of a C₇-C₃₉ fatty acid, an ester of a C₈-C₄₀ fatty alcohol, polyisobutene, a wax, a plant oil including coconut oil, almond oil and avocado oil, a mineral oil, a synthetic oil, and combinations thereof. In some aspects, the organic oil is a C₈-C₄₀ fatty alcohol, a C₁₀-C₄₀ fatty alcohol, a C₁₅-C₃₅ fatty alcohol, a C₂₀-C₃₀ fatty alcohol, or a C₂₅-C₃₀ fatty alcohol. In some aspects, the organic oil is an ester of a C₇-C₃₉ fatty acid, an ester of a C₁₀-C₃₅ fatty acid, an ester of a C₁₅-C₃₀ fatty acid, or an ester of a C₂₀-C₂₅ fatty acid. In some aspects, the organic oil is an ester of a C₈-C₄₀ fatty alcohol, an ester of a C₁₀-C₄₀ fatty alcohol, an ester of a C₁₅-C₃₅ fatty alcohol, an ester of a C₂₀-C₃₅ fatty alcohol, or an ester of a C₂₅-C₃₀ fatty alcohol. In some aspects, the organic oil is polyisobutene. In some aspects, the organic oil is a wax. In some aspects, the organic oil is a plant oil. In some aspects, the plant oil is coconut oil. In some aspects, the plant oil is almond oil. In some aspects, the plant oil is avocado oil. In some aspects, the organic oil is a mineral oil. In some aspects, the organic oil is a synthetic oil. In some aspects, the organic oil is derived from plants.

In some aspects, the non-volatile oil is an organic sunscreen ingredient selected from the group consisting of avobenzone, octisalate, oxybenzone, homosalate, octocrylene, octyl methoxycinnamate, 2-ethoxyethyl p-methoxycinnamate, ethylhexyl salicylate, ethylhexyl methoxycinnamate, and combinations thereof. In some aspects, the sunscreen ingredient is avobenzone. In some aspects, the sunscreen ingredient is octisalate. In some aspects, the sunscreen ingredient is oxybenzone. In some aspects, the sunscreen ingredient is homosalate. In some aspects, the sunscreen ingredient is octocrylene. In some aspects, the sunscreen ingredient is octyl methoxycinnamate. In some aspects, the sunscreen ingredient is 2-ethoxyethyl p-methoxycinnamate. In some aspects, the sunscreen ingredient is ethylhexyl salicylate. In some aspects, the sunscreen ingredient is ethylhexyl methoxycinnamate.

In some aspects, the non-volatile oil is selected from the group consisting of a conditioning agent, an emollient, a sunscreen oil, a natural oil, and combinations thereof. In some aspects, the non-volatile oil is a conditioning agent. In some aspects, the non-volatile oil is an emollient. In some aspects, the non-volatile oil is a sunscreen oil. In some aspects, the non-volatile oil is a natural oil.

### III. Personal Care Composition Ingredients

In some aspects, the personal care composition further comprises a silicone wax, an organic wax, or combinations thereof.

In some aspects, the personal care composition further comprises a silicone wax. In some aspects, the silicone wax is selected from the group consisting of C₃₀-C₄₅ alkyl dimethicone, C₃₀-C₄₅ alkyl cetearyl dimethicone crosspolymer, and combinations thereof.

In some aspects, the personal care composition further comprises an organic wax. In some aspects, the organic wax is selected from the group consisting of beeswax, ozokerite wax, carnauba wax, shea butter, stearic acid, cetyl alcohol, and combinations thereof.

In some aspects, the personal care composition further comprises an aqueous phase selected from the group consisting of water, glycerin, butylene glycol, propanediol, xanthan gum, steareth 2, steareth 21, sodium polyacrylate, disodium EDTA, hydroxyethyl urea, a cationic emulsifier, an anionic emulsifier, a non-ionic emulsifier, sodium chloride, and combinations thereof.

In some aspects, the personal care composition further comprises one or more of particulates, pigments and dyes. In some aspects, the particulates, pigments and dyes are selected from the group consisting of mica, magnesium carbonate, calcium carbonate, magnesium silicate, aluminum magnesium silicate, silica, talc, aluminum starch octenylsuccinate, polymethylsilsesquioxane, nylon-12, starch particles including tapioca starch and rice starch, cellulose particles, cellulose acetate particles, synthetic dyes including azo dyes, lakes, carmines and botanicals, iron oxide, zinc oxide, titanium dioxide, boron nitride, red iron oxide, yellow iron oxide, black iron oxide, organo-modified hectorite clay, polystyrene powder, silk powder, crystalline cellulose, titanated mica, iron oxide titanated mica, bismuth oxychloride, pearl, pearl mica, interference pigments, and combinations thereof.

In some aspects, the personal care composition further comprises one or more preservatives. In some aspects, the preservative is phenoxyethanol, ethylhexylglycerin, or combinations thereof.

In some aspects, the personal care composition further comprises one or more fragrances.

In some aspects, the personal care composition further comprises one or more humectants selected from the group consisting of propylene glycol, dipropylene glycol, polypropylene glycol, polyethylene glycol, sorbitol, hydroxypropyl sorbitol, hexylene glycol, glycerin, 1,3-butylene glycol, 1,2,6-hexanetriol, ethoxylated glycerin, propoxylated glycerin, and combinations thereof.

In some aspects, the personal care composition further comprises one or more resins. In some aspects, the resin is selected from the group consisting of a silicone resin, a hydrocarbon resin, an organic resin, a fluorinated resin, and combinations thereof.

In some aspects, the resin is a silicone resin comprising monofunctional siloxane units (M units), difunctional siloxane units (D units), trifunctional siloxane units (T units), quattrofunctional siloxane units (Q units), or combinations thereof. In some aspects, the resin is a silicone resin comprising monofunctional siloxane units (M units) having alkyl and/or aryl substituents on silicone atoms, difunctional siloxane units (D units) having alkyl and/or aryl substituents on silicone atoms, trifunctional siloxane units (T units) having alkyl and/or aryl substituents on silicone atoms, quattrofunctional siloxane units (Q units) having alkyl and/or aryl substituents on silicone atoms, or combinations thereof. In some aspects, the one or more resin is a silicone resin selected from the group consisting of MQ resin, MT resin, T-propyl resin, and combinations thereof.

In some aspects, the resin is an organic resin comprising homopolymers or co-polymers of vinyl polymers, acrylic polymers, methacrylic polymers, polyalkylenes, polyvinylpyrrolidones (PVP), or styrenic polymers, or combinations thereof.

In some aspects, the resin is an organic resin comprising polymeric gums, cellulose, cellulose derivatives, agar, pectin, gelatin, starch, starch derivatives, chitosan, chitosan derivatives, or combinations thereof. In some aspects, the polymeric gums are selected from the group consisting of alginates, carageenans, gum acacia, gum arabic, gum ghatti, gum karaya, gum tragacanth, guar gum, guar hydroxypropyltrimonium chloride, xanthan gum, gellan gum, dammar gum, olibanum gum, and combinations thereof. In some aspects, the cellulose derivatives are selected from the group consisting of sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropylcellulose, methyl cellulose, hydroxypropylmethyl cellulose, microcrystalline cellulose, and combinations thereof. In some aspects, the chitosan derivative is hydroxyethyl chitosan. In some aspects, the resin is a film former.

In some aspects, the personal care composition is a liquid, an emulsion, a lotion, a cream, an aerosol spray, a solid stick, a gel, a biphasic liquid, or a multi phasic liquid. In some aspects, the personal care composition is a deodorant, an antiperspirant, a skin cream, a facial cream, a hair shampoo, a hair conditioner, a mousse, a hair styling gel, a hair spray, a protective cream, a lipstick, a lip color, lip gloss, sunscreen, after sun lotion, sun spray, a facial foundations, blushes, makeup, mascara, a skin care lotion, a moisturizer, a facial treatment, eye serum, eye cream, a personal cleanser, a facial cleanser, a bath oil, a perfume, a shaving cream, a pre-shave lotion, an after-shave lotion, a cologne, a sachet, loose facial powder, compact powder, eye shadow, or a sunscreen.

### EXAMPLE 1

### Preparation of Alkane Mixture Examples 1-3 and Comparative Examples 1-2

The mixture of linear alkane of the invention is produced as outlined in EP2417199A1. Linear alkanes and branched C₁₀-C₁₂ alkane were combined and stirred until a homogeneous solution was formed. Table 1 lists the ingredients of alkane mixture examples 1-3 and comparative examples 1-2.

TABLE 1

| Composition | Example | Example | Example | Comparative | Comparative |
|---|---|---|---|---|---|
| Wt. % | 1 | 2 | 3 | Example 1 | Example 2 |
| Linear alkane (C₁₀) | 2.8 | 0 | 0.7 | | |
| Linear alkane (C₁₂) | 64.1 | 69.5 | 61.7 | 100 | |
| Linear alkane (C₁₄) | 3.4 | 1.3 | 2.1 | | |
| Linear alkane (C₁₆-C₁₈) | 0 | 2.4 | 4.8 | | |
| Branched alkane | 29.7 | 26.8 | 30.7 | | 100 |
| (C₁₀-C₁₂) | | | | | |

### EXAMPLE 2

### Preparation of Personal Care Formulation 1

Table 2 lists the ingredients of Personal Care Formulation 1 (hair oil). The procedure for preparing Personal Care Formulation 1 is described as following. Coconut oil and almond oil were combined and stirred at 50° C. until a homogeneous solution was formed. After cooling the mixture to 40° C., the alkane mixture example 2 was added to the mixture of coconut oil and almond oil. The product was stirred at room temperature until a homogeneous solution was formed. Personal Care Formulation 1 (hair oil) demonstrated enhanced playtime, no greasiness, better frizz reduction and better softness after 12 hrs as compared to a formulation without alkane mixture example 2.

TABLE 2

| Ingredient | Wt. % |
|---|---|
| Alkane Mixture Example 2 | 50% |
| Coconut Oil | 25% |
| Almond Oil | 25% |

### EXAMPLE 3

### Preparation of Personal Care Formulation 2

Table 3 lists the ingredients of Personal Care Formulation 2 (sunscreen). The procedure for preparing Personal Care Formulation 2 is described as following. Phase A ingredients were combined, heated, then stirred at 80° C. until a homogeneous solution was formed. Phase B ingredients were combined and stirred at 80° C. until a homogeneous solution was formed. Then phase B was slowly added to phase A while mixing to emulsification. The mixture was cooled to room temperature while stirring at 2000 rpm for 3 mins. Personal Care Formulation Example 2a was stirred at 5000 rpm for additional 2 mins. Personal Care Formulation Example 2b was stirred at 5000 rpm for additional 1 min.

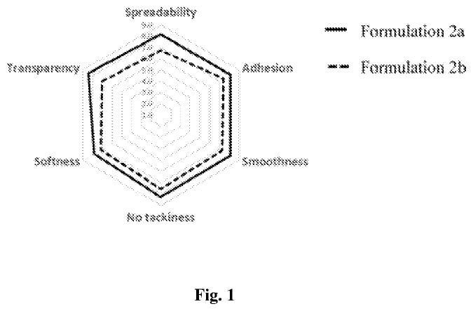

FIG. 1 shows a sensory panel study for Formulation Examples 2a and 2b. In this study, Formulation Example 2a shows better spreadability, skin adhesion, smoothness, softness, and transparency, and less tackiness compared to Formulation Example 2b containing Comparative Example 2.

TABLE 3

| | | | Formulation | Formulation |
|---|---|---|---|---|
| | | | Example 2a | Example 2b |
| Phase | Trade Name | INCI Name | (Wt %) | (Wt %) |
| A | Harmonie ™ Mesh | (Caprylic/Capric) | 2 | 2 |
| | Emulsifier | Triglyceride; | | |
| | | PEG/PPG-20/15 | | |
| | | Dimethicone | | |
| | Silsoft ™ 034 fluid | Caprylyl Methicone | 1 | 1 |
| | Alkane Mixture | Alkane Mixture | 19 | |
| | Example 2 | Example 2 | | |
| | Comparative | Comparative | | 19 |
| | Example 2 | Example 2 | | |
| | Serasense SF2 | Dimethicone | 9.0 | 9.0 |
| | Z-COTE HP1 | Zinc Oxide; | 9.0 | 9.0 |
| | | Triethoxycaprylylsilane | | |
| | Harmonie Luxe 4 | Silica | 4.0 | 4.0 |
| | powder | | | |
| | Velvesil ™ DM LC | Dimethicone; | 4.0 | 4.0 |
| | | Cetearyl Dimethicone | | |
| | | Crosspolymer | | |
| | SS4267 | Dimethicone; | 5.7 | 5.7 |
| | | Trimethylsiloxysilicate | | |
| | Uvinul MC80N | Ethylhexyl | 7.0 | 7.0 |
| | | Methoxycinnamate | | |
| B | Water | Water | 35.4 | 35.4 |
| | Butylene Glycol | Butylene Glycol | 2.0 | 2.0 |
| | NaCl | Sodium Chloride | 0.4 | 0.4 |
| | HISOLVE EPH | Phenoxyethanol | 0.6 | 0.6 |
| | NONION LT-20 | Polysorbate 20 | 0.9 | 0.9 |

### EXAMPLE 4

### Preparation of Personal Care Formulation 3

Table 4 lists the ingredients of Personal Care Formulation 3 (miracle hair oil). The procedure for preparing Personal Care Formulation 3 is described as following. Phase A ingredients were combined and stirred at room temperature until a homogeneous solution was formed.

TABLE 4

| Phase | Ingredient | INCI Name | Wt % |
|---|---|---|---|
| A | Alkane Mixture Example 3 | Alkane Mixture Example 3 | 63.2 |
| | Silsoft 8812 | Dimethicone; | 30 |
| | | Dimethiconol | |
| | Argan Oil | Argania Spinosa Kernel Oil | 5 |
| | SF1550 | Phenyltrimethicone | 1 |
| | SunShine Super Gold | Synthetic Fluorphlogopite; | 0.1 |
| | | Titanium Dioxide | |
| | Fragrance | Perfume | 0.2 |
| | Euxyl PE 9010 | Phenoxyethanol; | 0.5 |
| | | Ethylhexylglycerin | |

### EXAMPLE 5

### Preparation of Personal Care Formulation 4

Table 5 lists the ingredients of Personal Care Formulation 4 (after sun tanning lotion). The procedure for preparing Personal Care Formulation 4 is described as following. SilForm flexible resin and alkane mixture example 1 were combined and stirred at room temperature until a homogeneous solution was formed. All the other ingredients of phase A were added into the solution. The mixture was stirred at room temperature until a homogeneous solution was formed. Phase B ingredients were combined and stirred until a clear solution was formed. Then phase B was very slowly added to phases A while stirring at a high speed.

TABLE 5

| | | | Wt |
|---|---|---|---|
| Phase | Ingredient | INCI Name | % |
| A | Softouch* CCS102J | Boron Nitride | 6.00 |
| | SilForm* Flexible Resin | Polymethylsilsesquioxane | 0.50 |
| | Harmonie ™ Mesh | (Caprylic/Capric) | 5.00 |
| | Emulsifier | Triglyceride; | |
| | | PEG/PPG-20/15 | |
| | | Dimethicone | |
| | Alkane Mixture | Alkane Mixture | 14.50 |
| | Example 1 | Example 1 | |
| | SF1550 | Phenyltrimethicone | 1 |
| B | Deionized Water | Water | 69.00 |
| | Glycerin | Glycerin | 3.00 |
| | Sodium Chloride | Sodium Chloride | 1.00 |

### EXAMPLE 6

### Preparation of Personal Care Formulation 5

Table 6 lists the ingredients of Personal Care Formulation 5 (long wear lip color). The procedure for preparing Personal Care Formulation 5 is described as following. Phase A ingredients were combined and stirred at room temperature until a clear solution was formed. Phase B ingredients were combined and stirred at room temperature until a homogeneous solution was formed. Then phase B was added to phase A. The mixture was stirred at room temperature until a homogeneous solution was formed.

TABLE 6

| Phase | Ingredient | INCI Name | Wt % |
|---|---|---|---|
| A | SE 30 Gum | Dimethicone | 11.2 |
| | SF1550 | Phenyltrimethicone | 2.0 |
| | Alkane Mixture | Alkane Mixture | 20.0 |
| | Example 3 | Example 3 | |
| B | BENTONE GEL MIO | Mineral Oil | 6.1 |
| | V HV | Disteardimonium | |
| | | Hectorite, Propylene | |
| | | Carbonate | |
| | SR1000 resin | trimethylsiloxysilicate | 13.8 |
| | D&C Red 6 Ba Lake | CI 15850; | 24 |
| | CO Dispersion | Ricinus Communis | |
| | | [Castor] Seed Oil | |
| | Titanium Dioxide | Titanium Dioxide | 2.9 |
| | Alkane Mixture | Alkane Mixture | 20.0 |
| | Example 3 | Example 3 | |

### EXAMPLE 7

### Preparation of Personal Care Formulation 6

Table 7 lists the ingredients of Personal Care Formulation 6 (eye serum). The procedure for preparing Personal Care Formulation 6 is described as following. Phase A ingredients were combined and stirred at 70° C. until a homogeneous solution was formed. Phase B ingredients were combined and stirred at 70° C. until a homogeneous solution was formed. Phase B was added to phase A at 70° C. while stirring at 500 rpm. The mixture was stirred at 70° C. and 10000 rpm until it was homogeneous. Then the mixture was cooled to 50° C. with gentle stirring. Phase C ingredients were added into the mixture and stirred at 50° C. until a homogeneous solution was formed. Then the product was cooled to room temperature.

TABLE 7

| Phase | Ingredients | INCI Name | Wt % |
|---|---|---|---|
| A | Water | Aqua | up to 100 |
| | Glycerin | Glycerin | 3 |
| | HYDROVANCE | Hydroxyethyl Urea | 3 |
| | Moisturizing | | |
| | Agent | | |
| | Disodium EDTA | Disodium EDTA | 0.05 |
| B | HELIOFEEL | Glyceryl Stearate Citrate; | 4 |
| | | Polyglyceryl-3 Stearate; | |
| | | Hydrogenated Lecithin | |
| | Lecigel | Sodium Acrylates Copolymer; | 1 |
| | | Lecithin | |
| | Cetiol CC4 | Dicaprylyl Carbonate | 2 |
| | Cetiol 868 | Ethylhexyl Stearate | 3 |
| | Avocado Oil | Avocado (Persea Gratissima) Oil | 2 |
| | Shea butter | Butyrospermum Parkii (Shea | 2 |
| | | Butter) | |
| | BHT | Butylated Hydroxytoluene | 0.1 |
| | Alkane Mixture | Alkane Mixture | 5 |
| | Example 2 | Example 2 | |
| | Silsoft 034 fluid | Caprylyl Methicone | 2 |
| C | Silsoft E-Pearl | Water; | 3 |
| | PMF emulsion | Polymethylsilsesquioxane; | |
| | | Dimethicone; | |
| | | Isohexadecane; | |
| | | Cetearyl Methicone; | |
| | | PEG-40 Stearate; | |
| | | Steareth-2; | |
| | | Steareth-21 | |
| | Moist 24 | Imperata Cylindrica (Root Extract); | 1 |
| | | Aqua (water); | |
| | | Glycerin; | |
| | | PEG 8; | |
| | | Carbomer | |

### EXAMPLE 8

### Preparation of Personal Care Formulation 7

Table 8 lists the ingredients of Personal Care Formulation 7 (Air Feeling Hair Conditioner). The procedure for preparing Personal Care Formulation 7 is described as following. Phase A ingredients were combined and heated to about 80° C. Phase B was heated to about 80° C. Phases A and B were combined and homogenized for 3-5 mins, then cooled down to 50-60° C. with stirring. Phase C ingredients and phase D ingredients were added into the mixture of phases A and B, then homogenized for 3-5 min.

TABLE 8

| Phase | Ingredients | INCI Name | Wt % |
|---|---|---|---|
| A | Varisoft CT 85 | Behentrimonium Chloride | 3 |
| | TEGO Alkanol | Cetearyl Alcohol | 7 |
| | 1618 | | |
| B | Water | Aqua | 7 |
| C | Alkane Mixture Example 2 | Alkane Mixture Example 2 | 2.46 |
| | SF2900E | Dimethiconol | 0.54 |
| D | Silsoft* AM | Amodimethicone; | 3 |
| | 2222C PMF | Behentrimonium Chloride | |
| | emulsion | | |
| | PURAC HS 90 | Lactic Acid | 0.3 |
| | Preservative | Preservative | Q.S. |

### EXAMPLE 9

### Preparation of Personal Care Formulation 8

Table 9 lists the ingredients of Personal Care Formulation 8 (Leave on conditioning serum). The procedure for preparing Personal Care Formulation 7 is described as following. Phase A ingredients were combined and mixed well until homogeneous.

TABLE 9

| Phase | Ingredients | INCI Name | Wt % |
|---|---|---|---|
| Phase | CF1251 | Dimethicone | 20 |
| A | Alkane Mixture Example 3 | Alkane Mixture Example 3 | 80 |

1. A personal care composition comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more non-volatile oils selected from the group consisting of a silicone oil, a hydrocarbon oil, an organic oil, a fluorinated oil, and combinations thereof.

2. The personal care composition of claim 1, wherein the volatile alkane mixture comprises about 30% to about 85% by weight of linear alkanes having 9 to 18 carbon atoms relative to the total weight of the alkane mixture.

3. The personal care composition of claim 1, wherein the volatile alkane mixture comprises about 50% to about 75% by weight of linear alkanes having 9 to 18 carbon atoms relative to the total weight of the alkane mixture.

4. The personal care composition of any one of claims 1-3, wherein the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

5. The personal care composition of any one of claims 1-4, wherein the linear alkanes are of plant origin.

6. The personal care composition of any one of claims 1-5, wherein the linear alkanes are free of odd number carbon atoms.

7. The personal care composition of any one of claims 1-6, wherein the non-volatile oil is silicone with viscosity from about 10 cP to about 200,000,000 cP at 25° C.

8. The personal care composition of any one of claims 1-6, wherein the non-volatile oil is silicone with viscosity from about 500,000 cP to about 5,000,000 cP at 25° C.

9. The personal care composition of any one of claims 1-8, wherein the non-volatile oil is a silicone gum selected from the group consisting of polyalkylsiloxane, polyarylsiloxane, polyalkylsilanol, and combinations thereof.

10. The personal care composition of any one of claims 1-9, wherein the non-volatile oil is selected from the group consisting of an amino modified silicone, a polyether modified silicone, a phenyl modified silicone, and combinations thereof.

11. The personal care composition of any one of claims 1-10, wherein the non-volatile oil is a phenyl modified silicone selected from the group consisting of phenyltrimethicone, triphenyl dimethylvinyl disiloxane, phenylpropyldimethylsiloxysilicate, and combinations thereof.

12. The personal care composition of any one of claims 1-6, wherein the non-volatile oil is an organic oil selected from the group consisting of a C₈-C₄₀ fatty alcohol, an ester of a C₇-C₃₉ fatty acid, an ester of a C₈-C₄₀ fatty alcohol, polyisobutene, a wax, a plant oil including jojoba oil, argen oil, castor oil, coconut oil, almond oil and avocado oil, a mineral oil, a synthetic oil, and combinations thereof.

13. The personal care composition of any one of claims 1-6, wherein the non-volatile oil is a sunscreen ingredient selected from the group consisting of avobenzone, octisalate, oxybenzone, homosalate, octocrylene, octyl methoxycinnamate, 2-ethoxyethyl p-methoxycinnamate, ethylhexyl salicylate, ethylhexyl methoxycinnamate, and combinations thereof.

14. The personal care composition of any one of claims 1-13, wherein the non-volatile oil is selected from the group consisting of a conditioning agent, an emollient, a sunscreen oil, a natural oil, and combinations thereof.

15. The personal care composition of any one of claims 1-14, wherein the personal care composition further comprises a silicone wax, an organic wax, or combinations thereof.

16. The personal care composition of claim 15, wherein the silicone wax is selected from the group consisting of C₃₀-C₄₅ alkyl dimethicone, C₃₀-C₄₅ alkyl cetearyl dimethicone crosspolymer, and combinations thereof.

17. The personal care composition of claim 15, wherein the organic wax is selected from the group consisting of beeswax, ozokerite wax, carnauba wax, shea butter, stearic acid, cetyl alcohol, and combinations thereof.

18. The personal care composition of any one of claims 1-17, wherein the personal care composition further comprises an aqueous phase selected from the group consisting of water, glycerin, butylene glycol, propanediol, xanthan gum, steareth 2, steareth 21, sodium polyacrylate, disodium EDTA, hydroxyethyl urea, a cationic emulsifier, an anionic emulsifier, an non-ionic emulsifier, sodium chloride, and combinations thereof.

19. The personal care composition of any one of claims 1-18, wherein the personal care composition further comprises one or more of particulates, pigments and dyes.

20. The personal care composition of claim 19, wherein the particulates are selected from the group consisting of silica, talc, aluminum starch octenylsuccinate, polymethylsilsesquioxane, nylon-12, starch particles including tapioca starch and rice starch, cellulose particles, cellulose acetate particles, titanium dioxide, zinc oxide, boron nitride, mica, organo-modified hectorite clay, and combinations thereof.

21. The personal care composition of any one of claims 1-20, wherein the personal care composition is a liquid, an emulsion, a lotion, a cream, an aerosol spray, a solid stick, a gel, a biphasic liquid, or a multi phasic liquid.

22. The personal care composition of any one of claims 1-21, wherein the personal care composition is a deodorant, an antiperspirant, a skin cream, a facial cream, a hair shampoo, a hair conditioner, a mousse, a hair styling gel, a hair spray, a protective cream, a lipstick, a lip color, lip gloss, sunscreen, after sun lotion, sun spray, a facial foundations, blushes, makeup, mascara, a skin care lotion, a moisturizer, a facial treatment, eye serum, eye cream, a personal cleanser, a facial cleanser, a bath oil, a perfume, a shaving cream, a pre-shave lotion, an after-shave lotion, a cologne, a sachet, loose facial powder, compact powder, eye shadow, or a sunscreen.
